(19) United States
(12) Patent Application Publication
(10) Pub. No.: US 2017/0002428 A1
(43) Pub. Date: Jan. 5, 2017

(54) Detection and quantification of microRNAs in the circulation and the use of circulating microRNAs as biomarkers in cancer

(71) Applicant: National University of Ireland, Galway, Galway (IE)

(72) Inventors: Michael J. Kerin, Galway (IE); Nicola Miller, Galway (IE); Helen Heneghan, Tuam (IE)

(21) Appl. No.: 15/227,786

(22) Filed: Aug. 3, 2016

Related U.S. Application Data
(62) Division of application No. 14/313,829, filed on Jun. 24, 2014, now abandoned.
Application No. 14/313,829 is a continuation of application No. 13/046,502, filed on Mar. 11, 2011, now abandoned.
(60) Provisional application No. 61/313,059, filed on Mar. 11, 2010.

(51) Int. Cl. C12Q 1/68

(52) U.S. Cl. CPC C12Q 1/6886; C12Q 2600/112; C12Q 2600/158; C12Q 2600/178; C12Q 2600/118

(57) ABSTRACT

Biomarkers suitable for use in the diagnosis and prognosis of a number of cancers are identified and provided. Also, there are improved methods for the identification and quantification of such biomarkers in samples taken from patients.

### CROSS-REFERENCE TO RELATED APPLICATIONS

This application is a Divisional Application of U.S. application Ser. No. 14/313,829, filed Jun. 24, 2014, which is a Continuation Application of U.S. application Ser. No. 13/046,502, filed Mar. 11, 2011, which claims benefit of U.S. Provisional Application No. 61/313,059, filed Mar. 11, 2010, the contents of each of which are incorporated by reference in its entirety.

### FIELD OF THE INVENTION

The present invention relates to the identification of biomarkers suitable for use in the diagnosis and prognosis of a number of cancers. In addition, the invention relates to improved methods for the identification and quantification of such biomarkers in samples taken from patients.

### BACKGROUND OF THE INVENTION

Mi(cro)RNAs are short RNA molecules that regulate gene expression across a wide spectrum of biological and pathological processes. The discovery that mi(cro)RNA expression is frequently dysregulated in many disease processes has uncovered a new repertoire of molecular factors upstream of gene expression, which play critical regulatory roles in various cellular processes. In relation to cancer, aberrant miRNA expression has been shown to promote tumourigenesis, metastasis, and associate with other tumor characteristics. The finding that miRNA expression profiles have the capacity to accurately classify tumours according to existing clinicopathological variables has highlighted their potential as reliable prognostic indicators and cancer biomarkers.

The first evidence of involvement of miRNAs in malignancy came from the identification of a translocation-induced deletion at chromosome 13q14.3 in B-cell chronic lymphocytic leukemia 1. Loss of miR-15a and miR-16-1 from this locus results in increased expression of the antiapoptotic gene BCL2. Intensifying research in this field, using a range of techniques including miRNA cloning, quantitative PCR, microarrays and bead-based flow cytometric miRNA expression profiling, has resulted in the identification and confirmation of abnormal miRNA expression in almost every human malignancy including breast cancer (Table 1²).

TABLE 1
MiRNAs with altered expression in malignancy

| Tissue/ | | |
|---|---|---|
| tumor type | Increased expression | Decreased expression |
| Breast | miR-21, miR-29b-2 | miR-125b, miR-145 |
| | | miR-10b, miR-155, |
| | | miR-17-5p, miR-27b |
| Ovarian | miR-141, miR-200(a-c), | let-7f, miR-140, |
| | miR-221 | miR-145, miR199a, |
| | | miR-424 |
| Endometrial | miR-103, miR-107, | miR-99b, miR-152, |
| | miR-185, miR-205, | miR-193, miR-204, |
| | miR-210, miR-449 | miR-221, let-7i |
| Glioblastoma | miR-221, miR-21 | miR-181a, miR-181b, |
| | | miR-181c |
| Chronic | | miR-15, miR-16 |
| lymphocytic | | |
| leukemia | | |
| Lymphoma | miR-155, miR-17- | miR-15a |
| | 92cluster | |
| Colorectal | miR-10a, miR-17-92 | miR-143, miR-145, |
| | cluster, miR-20a, | let-7 |
| | miR-24-1, miR-29b-2, | |
| | miR-31 | |
| Thyroid | miR-221, miR-222, | |
| | miR-146, miR-181b, | |
| | miR-197, miR-346 | |
| Hepatocellular | miR-18, miR-224 | miR-199a, miR-195, |
| | | miR-200a, miR-125a |
| Testicular | miR-372, miR-373 | |
| Pancreatic | miR-221, miR-376a, | miR-375 |
| | miR301, miR-21, | |
| | miR-24-2, miR-100, | |
| | miR-103-1,2, miR- | |
| | 107, miR-125b-1 | |
| Cholangiocar- | miR-21, miR-141, | |
| cinoma | miR-200b | |
| Prostate | let-7d, miR-195, | miR-128a |
| | miR-203 | |
| Gastric | miR-223, miR-21, | miR-218-2 |
| | miR-103-2 | |
| Lung | mir-17-92 cluster, | let-7 family |
| | miR-17-5p | |

MiRNA expression has been observed to be up-regulated or down-regulated in tumours compared with normal tissue, supporting their dual role in carcinogenesis as either ‘oncomirs’ or tumour suppressors respectively. The focus of miRNA research to date has been at tumour tissue level; however recent reports on the detection of these molecules in the circulation has generated significant interest in the concept that systemic microRNAs hold potential as novel minimally invasive biomarkers for cancer and other disease processes. Methods of extracting miRNAs from the circulation, and subsequent quantification of systemic miRNA levels, are ill-defined. The techniques are variable and difficult to reproduce. Several questions permeate this field—which circulating medium is preferable for systemic miRNA investigations, which purification technique retrieves superior concentration of quality miRNA from blood, which concentration of miRNA is optimal for RQ-PCR analysis and which endogenous controls are appropriate for circulating miRNA studies.

The purpose of this investigation was to define a protocol for optimal extraction, quantification and analysis of miRNA expression in human blood samples. This was achieved using a breast cancer case-control cohort, to compare different methods for the extraction of microRNA from blood samples.

Current challenges in the management of breast cancer include a continuing search for sensitive and specific minimally invasive biomarkers that can be exploited to detect early neoplastic changes, thus facilitating the detection of breast cancer at an early stage, as well as for monitoring the progress of patients with breast cancer and their response to treatments. Existing diagnostic tools and biomarkers for breast cancer have many inherent deficiencies. Mammography is currently the gold standard diagnostic tool however it is not without limitations, including its use of ionizing radiation and a false negative rate of 8-10%. To date, only two markers are established in the routine evaluation of breast tumors: ER (for predicting response to endocrine therapies) and HER2/neu (for predicting response to Trastuzumab)³. Although these markers are assessed routinely, ER and HER2/neu assessment is far from perfect. A number of circulating tumor markers (e.g., carcinoembryonic antigen [CEA] and carbohydrate antigen 15-3 [CA 15-3]) are widely used in the management of breast cancer, but the sensitivity of these markers is low, and so they are not useful as screening tools although they have long been in clinical use as prognostic markers and to monitor for disease progression or recurrence⁴⁻⁶.

MiRNA expression studies in breast cancer indicate their importance and potential use as disease classifiers and prognostic tools in this field. A relevant and important feature of miRNAs is their remarkable stability. They are known to be well preserved in tissue samples even after years of formalin-fixation and paraffin-embedding, and can be efficiently extracted from and quantified in such specimens⁷. Investigation of cancer-specific miRNAs in the circulation is an emerging and exciting field of study. It is hypothesized that if miRNAs are present in the circulation of cancer patients, their unique stability and resilience should allow their detection and quantification to be practicable. The first report of circulating miRNAs, by Lawrie et al, described elevated serum levels of miR-21 in patients with diffuse large B-cell lymphoma⁸. Subsequently, circulating miRNAs have been postulated as novel biomarkers for cancer, and other disease processes. However this concept needs investigation to validate the theory. To date there has been no report on the role of circulating miRNAs in breast cancer.

The primary aim of our study was to investigate whether cancer specific miRNAs are detectable and altered in the circulation of breast cancer patients compared to age matched healthy controls, and if so, whether significantly altered systemic miRNAs reflected the tumor miRNA expression profile. We also aimed to identify the circulating medium which best represented miRNA levels (serum, plasma or whole blood). Previous studies, although few in number, have reported discrepancies between serum and plasma miRNA expression levels, or the investigators have chosen either medium alone for use in their studies⁹⁻¹¹. Finally a potential relationship between circulating miRNA levels and existing clinicopathological features of breast cancer such as tumor subtype, stage of disease, nodal status or hormone receptor status, was investigated.

### SUMMARY OF THE INVENTION

Described herein is a simple protocol for the extraction and quantification of microRNAs in the circulation. In particular there is provided an optimal extraction technique, which is most effective on whole blood specimens. Also provided are novel biomarkers for the detection of breast cancers. The ideal biomarker should be easily accessible such that it can be sampled relatively non-invasively, sensitive enough to detect early presence of tumors in almost all patients and absent or minimal in healthy tumor free individuals. The biomarker may be capable of indicating the presence of early stage breast cancers.

According to the present invention there is provided a diagnostic kit to detect cancers including breast cancer, diabetes, cardiovascular disease, hypercholesterolemia, obesity and the metabolic syndrome and liver toxicity, or to stratify patients according to expected prognosis comprising at least one oligonucleotide probe capable of binding to at least a portion of a circulating miRNA selected from the group comprising miR-16 (as endogenous control), miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, or miR-375 biomarkers.

A kit to detect cancers including breast cancer may comprise at least one oligonucleotide probe capable of binding to at least a portion of a circulating miRNA selected from the group comprising miR-16, miR-10b, miR-21, miR145, miR-155, miR-195, miR342, miR181c or let-7a biomarkers. The circulating miRNA may be selected from the group comprising miR-16, miR-195, miR342, miR181c and let-7a biomarkers. Such markers can be particularly useful as markers for early breast cancer. In particular embodiments the circulating miRNA may be miRNA-195 or let7a biomarkers. A kit for the detection of cancers including breast cancer may comprise at least one oligonucleotide probe capable of binding to at least a portion of a circulating miRNA selected from the group comprising miR-342 or miR-181c biomarkers.

A kit to detect or stratify metabolic diseases may comprise oligonucleotide probe capable of binding to at least a portion of a circulating miRNA selected from the group comprising miR-17-5p, miR-29a, miR-29b, miR-34a, miR-99a, miR-103, miR-122, miR-132, miR-143, miR-145, miR-375.

The kit may be adapted for performance of an assay selected from a real-time PCR assay, a micro-array assay, a histochemical assay or an immunological assay. For LRG assays cytochrome C may be used as a capturing ligand for building an ELISA. All such assays are well known to those of skill in the art. Where the assay is a histochemical assay, the antibody may be labelled with a suitable label. Suitable labels include coloured labels, fluorescent labels and radioactive labels.

The kit may be adapted to carry out a multiplex assay, in which a number of miRNAs are detected. The multiplex assay may be adapted to detect the miR-16 (as endogenous control), miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, and miR-375 biomarkers .

Alternatively the assay may be for the miR-16, miR-195, miR342, miR181c and let-7a markers. In another embodiment the assay may detect the miR-16, miR-10b, miR-21, miR145, miR-155, miR-195, miR342, miR18lc and let-7a biomarkers. In an alternative embodiment the multiplex assay may be for the detection of the miRNA-195 or let7a markers, or for the miR-342 or miR-181c markers. In still another embodiment the multiplex assay may be for the determination of the miR-17-5p, miR-29a, miR-29b, miR-34a, miR-99a, miR-103, miR-122, miR-132, miR-143, miR-145, miR-375 markers.

The kit is capable of detecting breast cancer, even in its earliest stage. The kit allows one to obtain prognostic information on the patient from their blood miRNA analysis—this information is currently obtained from the patient's clinical and/or pathological details, for example the size and grade of their tumour, hormone receptor status, presence of nodal or distant metastases. This information is then used to guide further treatment regimens. However the current methods of prognostication and stratification of breast cancers are far from perfect, so the miRNA blood test of the invention has the potential to improve the current system and be more accurate and specific in determining the patient's prognosis, and guiding adjuvant treatment regimens.

This novel diagnostic kit has potential for the following clinical applications:

(i) Breast screening: to identify changes in blood miRNA levels in cancer patients compared to normal cancer-free individuals, in an asymptomatic group.

(ii) Prognostication and prediction of outcome. This kit could identify differences between patients who present with early or late stage cancers, as well as stratify patients into molecular subtypes. This information can then aid in strategic planning of an individual patients therapeutic regimen.

(iii) Monitoring of response to treatments, through serial blood miRNA measurements using this kit; particularly in the neoadjuvant chemotherapy and metastatic disease settings.

The miRNAs identified and incorporated into this kit may also serve as novel therapeutic targets for breast cancer. The invention further provides a method of identifying a therapeutic agent capable of preventing or treating cancers including breast cancer, diabetes, cardiovascular disease, hypercholesterolemia, obesity and the metabolic syndrome and liver toxicity, comprising testing the ability of the potential therapeutic agent to reduce or enhance the expression of at least one protein selected from the group comprising miR-16 (as endogenous control), miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, or miR-375 biomarkers.

In another aspect the invention provides use of a circulating miRNA selected from the group comprising comprising miR-16 (as endogenous control), miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143 or miR-375 biomarkers to detect cancers including breast cancer, diabetes, cardiovascular disease, hypercholesterolemia, obesity and the metabolic syndrome and liver toxicity, or to stratify patients according to expected prognosis.

The detection may be carried out on a blood sample or a sample derived from blood.

The kit may be adapted for performance of an assay selected from a real-time PCR assay, a micro-array assay, a histochemical assay or an immunological assay. For LRG assays cytochrome C may be used as a capturing ligand for building an ELISA. All such assays are well known to those of skill in the art. Where the assay is a histochemical assay, the antibody may be labelled with a suitable label. Suitable labels include coloured labels, fluorescent labels and radioactive labels.

The invention also provides a method of detecting or screening for early stage breast cancers, comprising analysing a sample of blood taken from a patient for the presence of one or more biomarkers selected from the group comprising miR-16 (as endogenous control), miR-195, miR-342, miR-181c or let-7a, the increased expression of at least one of these miRNAs in the sample indicating the presence of breast cancer.

Also provided is a method of detecting small RNAs (that is molecules of less than about 200 nucleotides) in the circulation comprising:—

(i) Treating whole blood with TRIZOL® reagent at a ratio of one part blood to three parts TRIZOL® reagent,

(ii) Adding 1-bromo-4-methoxybenzyene (also known as bromoanisole,

(iii) Centrifuging the mixture at at least 12000 g for at least 5 min at less than 10° C.

(iv) Precipitating RNA from the aqueous phase

(v) quantifying the miRNAs precipatated.

The ratio of 1-bromo-4-methoxybenzyene to blood or blood-derived sample is suitably in the range of 0.2-0.8 ml 1-bromo-4-methoxybenzyene to 1 ml blood or blood-derived sample. Preferably 0.2 ml 1-bromo-4-methoxybenzyene to 1 ml blood or blood-derived sample is used.

It is important to separate phases at a cool temperature (4-10° C.]—centrifugation performed at elevated temperatures may sequester DNA into the aqueous phase. Thus preferably the mixture is centrifuged at 4° C. for 15 min.

Optionally, following the addition of 1-bromo-4-methoxybenzyene, 8-32 microlitres of polyacryl carrier may be added to the blood/TRIZOL®/1-bromo-4-methoxybenzyene solution. This augments visualization of the RNA pellet once it is precipitated.

Any RNA precipitation method could be used (e.g. Sodium acetate, Isopropanol, etc) as would be known to those skilled in the art. Particularly preferred is use of one part Isopropanol to one part aqueous RNA solution, centrifuged for 8 minutes at 12,000 g, at a higher temp of 18 degree Celsius.

The precipitated RNA may then be washed with ethanol, preferably twice. The second wash with ethanol does improve the purity of the RNA isolated.

Quantification of miRNAs may be carried out via NanoDrop® spectrophotometry set at a conversion factor of 33 μg/ml or equivalent spectrophotometry, or Agilent quantification method.

The method may further comprise synthesising cDNA on 1-1000 ng of small RNA, as quantified by the method described above. The small RNA is reverse transcribed using stem loop RT primers, specific for each miRNA target and diluted with nuclease-free water to give 50 μM concentration per reaction, according to standard terms and conditions. The DNA may then be stored at about −20° C.

The miRNA expression levels may be quantified (relative quantification) by real-time PCR, using the expression level of miR-16 and/or another stably expressed small RNA(s) to normalise the expression level of the target miRNA. All reactions may be performed in triplicate and using an interassay control. The data may be analysed using 2⁻ ΔΔCT to determine relative quantities of the target miRNA.

The kits, assays and methods of the invention may comprise determining the level of at least 2 miRNA biomarkers from the group, or at least 3 biomarkers, or at least 4 biomarkers, or at least 5 biomarkers, or at least 6 biomarkers from the group. In other embodiments the invention provides determining the level of all biomarkers from the group, or at least 10 biomarkers or at least 12 biomarkers from the group.

A kit to be applied to the detection of metabolic diseases would require quantification of miRNAs which represent biomarkers of metabolic diseases including miR-17-5p, miR-29a, miR-29b, miR-34a, miR-99a, miR-103, miR-122, miR-132, miR-143, miR-145, miR-375.

The development of a miRNA primer/probe signature panel to rapidly test blood samples for expression levels of candidate metabolic miRNAs allows the kit to be used to predict obese patients who are at high risk of developing the metabolic syndrome, those who would benefit most from bariatric surgery as well as blood lipid and glucose levels. This in turn allows the development of therapeutic strategies using miRNA replacement or antagonism for the treatment of obesity and the metabolic syndrome. In particular, replacement of miR-17-5p in obese patients may have the potential to restore catabolic activity and thus aid in weight loss. Similarly, replacement of miR-143 in obese diabetic patients may revert their glycaemic indices to normal, and thus cure obese (and possibly non-obese) patients of diabetes.

A kit to be applied to the detection of cancers would require quantification of miRNAs which represent biomarkers of miR-16 (as endogenous control), miR-10b, miR-21, miR145, miR-155, miR-195, miR342, miR181c or let-7a.

Circulating miR-195 is a marker for early stage breast cancer; increased miR-195 levels in blood are observed in breast cancer patients. More significantly increased circulating miR-195 levels are a marker of more advanced breast cancers (compared with early stage tumours) so circulating miR-195 levels may be an indicator of stage of disease. MiR-195 in combination with let-7a, miR-342 and miR-181c may be an indicator of subtype of breast cancer, although alone, miR-195 does not indicate subtype of breast cancer. Circulating miR-195 levels decreased 2 weeks following tumour resection, therefore circulating miR-195 may be a marker for determining complete surgical resection/response to therapy/recurrence of disease. Thus an inhibitor of miR-195 may also have a therapeutic application.

Circulating let-7a is a marker for early stage breast cancer as increased levels of this marker are seen in circulation of breast cancer patients. Circulating let-7a is a marker for pre-invasive breast cancer, which is the earliest stage of breast cancer. The present inventors have found that let-7a, one of the most well established and defined cancer associated miRNAs, was significantly elevated in the circulation of patients with several visceral malignancies (breast, prostate, colon and renal cancers) compared to controls. The findings are in keeping with existing evidence which supports let-7a as a protagonist in many cancers, particularly lung, breast, colon, gastric and ovarian. However the present study observed a paradoxical effect in the circulation (i.e. a significant increase in systemic let-7a levels in cancer patients compared to controls), to that described previously at tumour tissue level where let-7a is most commonly found to be under-expressed in tumour tissue compared to normal tissue, for these individual cancers.

Circulating miR-21 & miR-10b are increased in oestrogen-receptor negative disease so these have potential as markers for a subtype of disease, in addition to measuring miR-195, let-7a, miR-342 and mir-181c levels. Circulating miR-21 serves as a marker for disease progression, and advanced or aggressive disease.

According to certain aspects of the disclosures, a method for detecting or screening for breast cancer or a metabolic disease in a patient is provided. The method may comprise obtaining a sample comprising whole blood from the patient, treating the sample with TRIZOL® reagent, adding 1-bromo-4-methoxybenzyene to the sample with TRIZOL® reagent to provide a mixture, centrifuging the mixture at at least 12000 g for at least 5 min at less than 10° C., so as to provide an aqueous phase and a non-aqueous phase, precipitating RNA from the aqueous phase, and quantifying one or more miRNAs precipitated, wherein the one or more miRNAs are selected from the group consisting of miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, and miR-375 biomarkers, and wherein a statistically significant increase of at least one of said miRNAs relative to a level of said miRNA in control samples from healthy individuals is indicative of breast cancer or metabolic disease in the patient.

In some embodiments, in the method, the one or more miRNAs may be selected from the group consisting of miR-10b, miR-21, miR145, miR-155, miR-195, miR342, miR181c, let-7a, miR-29a, miR-29b, miR-34a, miR-99a, miR-103, miR-122, miR-132, miR-143, and miR-375 biomarkers. In some other embodiments, miR-16 may be also quantified as an endogenous control.

In certain embodiments, the method is for detecting or screening for early stage breast cancer in the patient, and the one or more miRNAs may be selected from the group consisting of miR-195, miR-342, miR-181c and let-7a, wherein a statistically significant increase of at least one of miR-195, miR-342, miR-181c or let-7a relative to a level of said miRNA in control samples from healthy individuals is indicative of breast cancer in the patient.

In alternative embodiments, the method is for detecting or screening for a metabolic disease, and the one or more miRNAs are selected from the group consisting of miR-17-5p, miR-29a, miR-29b, miR-34a, miR-99a, miR-103, miR-122, miR-132, miR-143, miR-145, and miR-375, wherein a statistically significant increase of at least one of miR-17-5p, miR-29a, miR-29b, miR-34a, miR-99a, miR-103, miR-122, miR-132, miR-143, miR-145, or miR-375 relative to a level of said miRNA in control samples from healthy individuals is indicative of metabolic disease in the patient. In some of certain embodiments, the one or more miRNAs are selected from the group consisting of miR-29a, miR-29b, miR-34a, miR-99a, miR-103, miR-122, miR-132, miR-143, miR-145, and miR-375.

In some embodiments, in the method, the ratio of whole blood to TRIZOL® reagent is one part whole blood to three parts TRIZOL® reagent.

In some other embodiments, in the method, the ratio of 1-bromo-4-methoxybenzyene to the sample comprising the whole blood is in the range of 0.2-0.8 ml 1-bromo-4-methoxybenzyene to 1 ml the sample.

In still some other embodiments, in the method, the ratio of 1-bromo-4-methoxybenzyene to the sample comprising the whole blood is 0.2 ml 1-bromo-4-methoxybenzyene to 1 ml of the sample.

In still some other embodiments, in the method, following the addition of 1-bromo-4-methoxybenzyene, a polyacryl carrier is added to the mixture comprising the whole blood, TRIZOL® reagent, and 1-bromo-4-methoxybenzyene.

In still some other embodiments, in the method, the RNA is precipitated by addition of isopropanol to the aqueous phase. In some of such embodiments, one part isopropanol to one part aqueous phase (v/v) is added.

In still some other embodiments, the method may further comprise centrifuging the aqueous phase containing the precipitated RNA for 8 minutes at 12,000 g, at 18 degree Celsius.

In still some other embodiments, in the method, the precipitated RNA is washed with ethanol, preferably twice.

In still some other embodiments, in the method, quantification of miRNAs is carried out via NanoDrop® spectrophotometry set at a conversion factor of 33 μg/ml or equivalent spectrophotometry.

In still some other embodiments, in the method, quantification of miRNAs is carried out via the Agilent quantification method.

In still some other embodiments, the method may further comprise synthesizing cDNA of said one or more quantified miRNAs. In some of such embodiments, the cDNA is synthesized from 1-1000 ng of the one or more quantified miRNAs. In certain embodiments, the one or more of the quantified miRNAs is reverse transcribed using stem loop RT primers, specific for each miRNA target and diluted with nuclease-free water to give 50 μM concentration per reaction.

In still some other embodiments, in the method, miRNA expression levels are relatively quantified by real-time PCR, using an expression level of miR-16 and/or another stably expressed miRNA(s) to normalize an expression level of a target miRNA.

In still some other embodiments, the method may utilize at least one oligonucleotide primer and/or probe capable of binding to at least a portion of a circulating miRNA selected from the group comprising miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, and miR-375 biomarkers. The circulating miRNA may be selected from the group comprising miR-16, miR-195, miR342, miR181c and let-7a biomarkers. Such markers can be particularly useful as markers for early breast cancer. In particular embodiments the circulating miRNA may be miRNA-195 or let7a biomarkers. In certain embodiments, the method may utilize at least one oligonucleotide primer and/or probe capable of binding to at least a portion of a circulating miRNA selected from the group comprising miR-342 or miR-181c biomarkers. In certain some embodiments, the oligonucleotide primer and/or probe may be labelled with a suitable label. Suitable labels include coloured labels, fluorescent labels and radioactive labels.

### BRIEF DESCRIPTION OF THE DRAWINGS

FIG. 1: MiR-16 expression in whole blood samples of 127 subjects: 83 samples were from women with histologically confirmed breast cancer and from whom the blood sample was taken preoperatively, whilst 44 samples were from disease free and age matched control females. This illustrates the finding that miR-16 is stably expressed across all analyzed blood samples and thus is a suitable endogenous control.

FIG. 2: Phase separation of whole blood using Tri Reagent® BD copurification protocol. Following centrifugation of a homogeneous mixture of blood (1 ml, unclotted), TRIZOL® (3 ml) and Bromoanisole (200 μl), the mixture separates into 3 phases: the upper clear aqueous phase containing RNA exclusively, a DNA interphase, and the lower organic phase containing protein and lipids.

FIG. 3: Expression levels of miR-195 (A) and let-7a (B) in blood samples from breast cancer patients including preoperative (n=83) and postoperative (n=29) patients and controls (n=44). At two weeks postoperatively a significant decrease in mean circulating miR-195 and let-7a levels was observed, reaching levels comparable with control subjects.

FIG. 4: MiR-195 expression in breast cancer tissues (n=65), tumor associated normal (n=18), preoperative invasive breast cancer blood samples (n=73) and healthy control bloods (n=44). Mir-195 expression in tumor tissue differed significantly to TAN (p<0.001) and similarly its expression in blood from breast cancer patients differed significantly to healthy control blood (p<0.001).

FIG. 5: MiR-195 expression according to stage of disease, in invasive breast cancer tissue specimens (n=65) and invasive breast cancer preoperative blood sample (n=73) and healthy control blood (n=44). Tumor miR-195 expression was significantly higher in Stage IV compared to Stages I & II tumors (p=0.006 and 0.039 respectively ANOVA and Tukey post hoc analysis). Whilst there was no significant difference between stage of disease and miR-195 expression in blood from breast cancer patients, there was a similar trend for increasing miR-195 levels with advancing stage of disease.

FIG. 6: Correlation of miR-195 levels in blood (n=65) and tumor tissue (n=65) from stage-matched invasive breast cancer patients. [Stage 1 (n=13), Stage 2 (n=29), Stage 3 (n=17), Stage 4 (n=6)]. There was no significant correlation between blood and tumor miR-195 levels, for stages of disease. (Pearson's correlation coefficient r=0.126, p=0.323).

FIG. 7: MiR-195 expression in invasive breast cancer tissue (n=65), according to HER2/neu status. HER2/neu positive tumors (n=20) expressed higher miR-195 than HER2/neu negative tumors (n=45), p=0.002.

FIG. 8: Decreased circulating levels of miR-195 in 2-week post-operative blood samples from 29 patients. The box-plot indicates pre-operative miR-195 levels which dropped to levels comparable with the control group following curative tumour resection.

FIG. 9: miRNA signature of miR-195, miR-181c, miR-342 and let-7a which are specific for breast cancer.

### DETAILED DESCRIPTION OF THE DRAWINGS

### Methods

Blood collection: Venous blood samples (non-fasting) were collected as follows: from each participant, whole blood was collected in two Vacuette EDTA K3E blood bottles (Grenier Bio-one); one processed for plasma and the other unprocessed; and a third sample was collected in Vacutainer Serum Separator Tubes II (Becton Dickinson) for serum. Upon obtaining, samples for serum collection were allowed to clot for 30 min and then all samples destined for serum and plasma collection were centrifuged at 2000 rpm@4° C. for 10 minutes in a Sorvall RT6000D centrifuge. Plasma/serum was removed, aliquoted and stored at −20° C. until required. The unprocessed whole blood sample was stored at 4° C. until required.

### RNA Isolation:

### Protocol 1.

Separate purification of miRNA and larger RNAs (>200 nt) using column based method:

Micro RNA was extracted from whole blood, serum and plasma samples as follows: In brief 250 μl of thawed serum/plasma was mixed with 1 ml QIAzol® lysis reagent and 250 μl of Chloroform, and then centrifuged at 12000 g for 15 min at 4° C. Large RNA fractions (>200 nucleotides) and small RNA fractions (<200 nucleotides) were isolated separately using the QIAGEN RNeasy Minikits and the protocol supplied by the manufacturer.

### Protocol 2. Copurification of Total RNA:

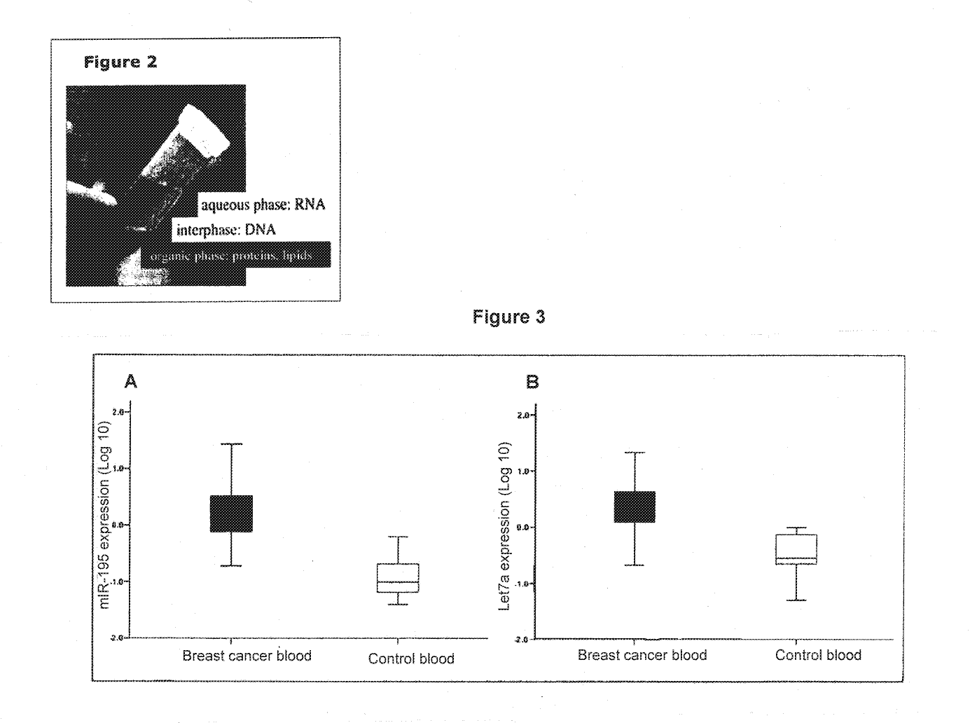

Total RNA was extracted from whole blood, serum and plasma samples using a modification of the TRI REAGENT® BD (Molecular Research Centre, Inc., Cincinnati, Ohio) copurification protocol, as follows: Using 1 ml of whole blood, or its derivatives, phase separation was performed by the addition of 3 ml of TRIZOL® reagent (Invitrogen, Carlsbad, Calif.) and 200 microlitres of 1-bromo-4-methoxybenzene to augment the RNA phase separation process (FIG. 2). The homogenous blood/TRIZOL® mixture was split between two 2 ml collection tubes and then centrifuged at 12000 g for 15 min, at 4° Celsius. The clear aqueous phase (approximately 1 ml) from each tube was then removed, transferred to fresh collection tubes respectively and RNA precipitated by the addition of 1 ml of Isopropanol and centrifugation of the solution at 12000 g for 8 min at 18° C. Following removal of the supernatant, the RNA pellet was then washed with 1 ml of 75% ethanol. We performed an additional ethanol wash to improve the purity of RNA isolated, which was reflected in an improved 260/280 ratio. Each RNA pellet was briefly air dried and then solubilised using 30 microlitres of nuclease free water. Hence each 1 ml of whole blood yielded 60 microlitres of total RNA when the two matched RNA pellets were solubilised, mixed together again, and finally transferred to storage tubes prior to storage at −80° Celsius.

### RNA Isolation

Total RNA was extracted from 1 ml of blood/serum/plasma respectively using TRI Reagent® BD (Molecular Research Centre, Inc., USA). RNA was extracted from breast tissue as described above. RNA concentration and integrity were determined using by NanoDrop spectrophotometry (NanoDrop® ND-1000 Technologies Inc, DE, USA) and an Agilent Bioanalyzer (Agilent Technologies, Germany), respectively.

### Assessing Concentration and Integrity of RNA

RNA concentration was determined using the Nanodrop® ND-1000 Spectrophotometer (NanoDrop Technologies Inc, Wilmington, Del., USA). The wavelength-dependent extinction coefficient ‘33’ was taken to represent the micro component of the RNA solution. In general we obtained concentrations ranging between 30-300 nanograms per microlitre of miRNA. Integrity was assessed using the RNA 6000 Nano LabChip Series II Assay with the 2100 Bioanalyzer (Agilent Technologies, Waldbronn, Germany).

Analysis of miRNA Gene Expression

We chose to study a panel of nine miRNAs in the circulation of breast cancer patients (miR-10b, miR-21, miR-145, miR-155, miR-195, miR-181c, miR-342, let 7a, and miR-16). These were chosen for their potential as breast cancer biomarkers based on their documented association with breast cancer in existing literature, and from ongoing work in our Department of Surgery investigating the role of microRNAs in breast cancer.

RQ-PCR quantification of miRNA expression was performed using TaqMan MicroRNA® Assays (Applied Biosystems, Foster City, Calif., USA) according to the manufacturer's protocol. Total RNA (100 ng) was reverse-transcribed using the MultiScribe™-based High-Capacity cDNA Archive kit (Applied Biosystems, Foster City, Calif., USA). RT-negative controls were included in each batch of reactions. PCR reactions were carried out in final volumes of 10 μl using an ABI 7900 HT Fast Real-Time PCR System (Applied Biosystems, Foster City, Calif., USA). Briefly, reactions consisted of 0.7 μl cDNA, 5 μl TaqMan® Universal PCR Fast Master Mix, 0.2 μM TaqMan® primer-probe mix (Applied Biosystems, Foster City, Calif., USA). Reactions were initiated with 10-minute incubation at 95° C. followed by 40 cycles of 95° C. for 15 seconds and 60° C. for 60 seconds. An interassay control derived from a breast cancer cell line (ZR-75-1) was included on each plate and all reactions were performed in triplicate. MiR-16 was used as endogenous controls to standardize miRNA expression. The threshold standard deviation for intra-assay and inter-assay replicates was 0.3. The percentage PCR amplification efficiencies (E) for each assay were calculated, using the slope of the semi-log regression plot of cycle threshold versus log input of cDNA (10-fold dilution series of five points), with the following equation:

E=(10−1/slope−1)×100

A threshold of 10% above or below 100% efficiency was applied. The relative quantity of miRNA expression was calculated using the comparative cycle threshold (ΔΔCt) method¹², normalised to miR-16 levels, and the lowest expressed sample was used as a calibrator.

### Study Cohort

Following ethical approval and written informed consent, blood samples (whole blood, serum and plasma) were collected prospectively from 127 females, including 83 consecutive breast cancer patients and 44 healthy age-matched female volunteers who served as controls for this study. All patients had histologically confirmed pre-invasive or invasive breast cancer and their relevant demographic and clinicopathological details were obtained from our prospectively maintained breast cancer database. The histological tumor profile of patients in this study reflects that of a typical breast cancer cohort, with the majority of invasive tumors being of ductal type, and Luminal A epithelial subtype (Table 1). In addition, repeat blood samples were collected from a subset of this cohort, at their initial clinical review two weeks postoperatively (n=29). The control blood samples were collected from healthy women with no current or previous malignancy, or inflammatory condition. A similar cohort of age and stage-matched breast cancer patients (n=65) were identified from whom tumor and tumor associated normal (TAN) tissues were prospectively collected (Table 1).

### MiRNA Targets

The expression of a panel of 9 cancer associated miRNAs was chosen on the basis of their reported relevance to breast cancer (Table 2)^{10 13-16}.

### Statistical Analysis

Data were analysed using the software package SPSS 17.0 for Windows. Due to the magnitude and range of relative miRNA expression levels observed, results data were log transformed for analysis. Data are presented as Mean±SD. There was no evidence against Normality for the log transformed data as confirmed using the Kolmogorov-Smirnov test. The two-sample t-test was used for all two sample comparisons and ANOVA, followed by Tukey HSD Post Hoc test, to compare the mean response between the levels of the between subject factors of interest. All tests were two tailed and results with a p<0.05 were considered statistically significant.

TABLE 2
Candidate miRNAs for investigation in the
circulation of breast cancer patients.

| miRNA of | |
|---|---|
| interest | Previous association with breast cancer |
| miR-10b | Decreased expression in breast tumour tissue compared |
| | to normal breast tissue |
| miR-21 | Increased expression in breast tumour tissue compared |
| | to normal breast tissue. Also increased in other solid |
| | cancers: colorectal, pancreas, gastric, lymphomas |
| miR-145 | Decreased expression in breast tumour tissue compared |
| | to normal breast tissue |
| miR-155 | Increased expression in breast tumour tissue compared |
| | to normal breast tissue |
| miR-195 | Reported by Mattie et al to be associated with hormone |
| | receptor status, as part of a ‘miRNA signature’ |
| let 7a | Reliable endogenous controls for analysis of miRNA by |
| | RQ-PCR in human breast tissue, with miR-16 |
| miR-16 | Reliable endogenous controls for analysis of miRNA by |
| | RQ-PCR in human breast tissue, with let-7a and used |
| | as a single endogenous control for investigating serum |
| | miRNA levels in recent studies. |
| miR-342 | Increased expression in breast tumour tissue compared |
| | to normal breast tissue, and expression correlates |
| | with hormone receptor status (ER) |
| miR-181c | Increased expression in breast tumour tissue compared |
| | to normal breast tissue and expression correlates |
| | with hormone receptor status (ER) |

### Results

Comparison of Techniques: Separate miRNA Purification vs. Total RNA Copurification

The standard protocol for isolation of miRNA from whole blood, serum and plasma, using QIAGEN RNeasy Minikits, yielded low concentrations of miRNA (Range 1.9-86.1 ng/μl, for 25 samples). Applying the modified Tri Reagent® BD (Molecular Research Centre, Inc., Cincinnati, Ohio) copurification protocol to patients matched blood, serum and plasma samples, we obtained consistently higher concentrations of miRNA with this method, in particular for whole blood (Range 20.3-221.6 ng/μl, for 110 samples, Table 3). The concentration and integrity of the miRNA in these samples was confirmed using the RNA 6000 Nano LabChip Series II Assay with an Agilent Bioanalyzer. Additionally, RT-PCR quantitation of miRNA extracted from blood (whole, serum and plasma) samples using the latter technique, indicated that earlier amplification, therefore higher yields, of miRNAs were observed in whole blood compared to matched serum and plasma (Table 4).

TABLE 3
Comparison of RNA isolation techniques
according to miRNA yield (ng/μl)

| | Whole blood | Serum | Plasma |
|---|---|---|---|
| | miRNA | miRNA | miRNA |
| Qiagen separate purification | | | |
| Patient #3038 | 6.75 | 16.79 | 48.41 |
| Trizol copurification | | | |
| Patient #3038 | 139.3 | 121.3 | 135.7 |

TABLE 4
CT values for miR-16 expression in matched whole blood,
serum and plasma (100 ng miRNA per reaction)

| | Whole blood | Serum | Plasma |
|---|---|---|---|
| Patient 1 | 15.7 | 31.3 | 31.6 |
| Patient 2 | 17.9 | 33.1 | 30.9 |
| Patient 3 | 17.4 | 32.7 | 32.9 |

Whole blood holds advantages over serum or plasma, as a medium for RNA isolation; most notably that no additional processing of the sample is required prior to RNA extraction and therefore is less labour intensive to work with.

It is not known why the separate purification of miRNAs from blood and its derivatives results in lesser yields of miRNA. Whilst this method works effectively for tissue miRNA isolation, results are less successful when it is applied to fluid media such as blood, serum or plasma. One potential explanation may be that the silica-membrane of the RNeasy spin columns, with its maximal binding capacity of 100 μg RNA¹⁷, may not have the capacity to bind the maximal miRNA component in a small volume of a dilute circulating medium such as blood. Perhaps modification of the column membrane may alter its affinity for miRNA molecules in fluid specimens and improve the ability of this technique to yield higher quantities of quality miRNA. Another potential explanation of why the separate purification protocol using QIAGEN RNeasy Minikits yields low miRNA concentration, is that the recommended volume of blood/serum/plasma (250 μl) to be filtered through the columns contains only dilute miRNA amounts. If miRNAs in the circulation represent what is shed from the tumour or released from lysed tumour cells, then one may hypothesize that the miRNA complement in the entire blood volume is at much lower concentration than at tissue level. By sampling only a tiny volume of the circulation such as 250 μl, the miRNA concentration within such a small volume could be expected to be very low. Perhaps if the columns and their silica membranes were modified to hold larger blood volumes such as 1 ml, then the resulting miRNA yield could be increased.

Whole Blood miRNA Yield Superior from Fresher Samples

Our comparative analysis of whole blood samples which had been stored for varying lengths of time, indicated that fresher blood samples yielded higher concentrations than blood stored for several weeks or months (up to 12 months) (Table 5).

TABLE 5
Concentrations (ng/ul) of miRNA in fresh and stored whole blood

| | Fresh* | Stored for 6 weeks | Stored for 6 months |
|---|---|---|---|
| Patient 1 | 81.59 | 28.4 | 23.1 |
| Patient 2 | 122.4 | 23.88 | 19.8 |
| Patient 3 | 170.9 | 66.46 | 28.5 |

*RNA isolated from sample within 3 days of blood collection

Whilst miRNAs are remarkably stable molecules in tissue and circulation¹⁰, it would still appear that storing the blood samples for lengthy periods of time allows a minor degree of miRNA degradation. In order to maximize the yields from a small volume of blood, we recommend isolation of the miRNA shortly after the sample as been obtained. Nevertheless, it must be noted that even in whole blood samples stored at 4° C. for several months; it is still possible to accurately and consistently quantify the miRNA complement of the sample by RQ-PCR. The primary advantage then of prompt RNA isolation from blood is a greater quantity of miRNA which allows a greater number of miRNA targets to be measured from a single 1 ml aliquot of blood.

Concentration of miRNA per PCR Reaction

The concentration of miRNA per cDNA synthesis reaction which returned superior RQ-PCR amplification was 10Ong per reaction, compared to when the starting miRNA concentration per was 5 ng, 10 ng or 50 ng (Table 6).

TABLE 6
miR-16 amplification (CT cycle) using differing
concentrations of miRNA per reaction

| | 100 ng | 50 ng | 10 ng | 5 ng |
|---|---|---|---|---|
| Patient 1 | 15.7 | 16.9 | 23.1 | 24.6 |
| Patient 2 | 17.9 | 19.3 | 23.8 | 24.9 |

Over the last few years, tissue miRNA quantitation by RQ-PCR has developed substantially. High throughput technology and currently available primers and probes have advanced dramatically so that the technique is sensitive enough to quantify miRNAs from a minute starting volume of RNA. This is hugely beneficial when the specimen is limited in size, as is routinely the case with small tumour biopsy specimens. Hence it has become routine to use RNA quantities as low as 5 ng for tissue miRNA quantitation. However the same limitation does not apply to blood based miRNA investigations, as the sample is easier to obtain in larger quantities, and can be resampled at various time-points without great difficulty or inappropriate distress to the patient. Therefore it is more feasible to use starting quantities as high as 100 ng per reaction for circulating miRNA investigations, and in order to return superior miRNA expression results, this is to be recommended based on the data presented here. The concentrations of miRNA used in existing reports of circulating miRNA investigations have varied between 5-50 ng. The authors have not described their reasoning behind choosing specific concentrations, or indeed if they made any effort to determine which concentration is optimal. This is the first study to address this specific and important methodological concern.

Endogenous Control for Circulating miRNA Investigations

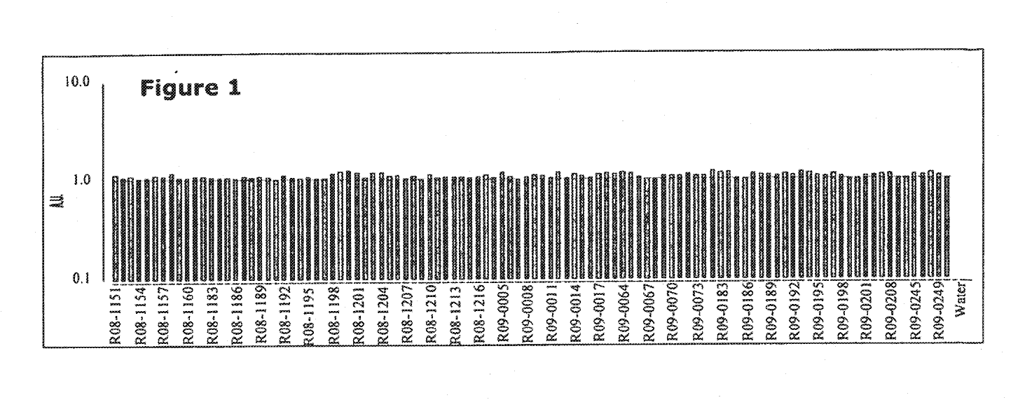

MiR-16 was found to be abundantly expressed in all samples included in this study; in cancer patients and healthy controls alike. Thus miR-16 was used as the endogenous control to normalise RQ-PCR data (FIG. 1). There is no consensus in the published reports on blood-based miRNA studies as to what the ideal endogenous control is for these investigations. However miR-16 has been shown to be abundantly expressed in normal healthy individuals and levels in the circulation have been documented several times to be unaltered in the presence of malignancy. Hence circulating miR-16 is the most commonly used miRNA reference gene in this context to date⁸⁻¹⁰.

### Detection of MiRNA in the Circulation of Breast Cancer Patients

Expression of nine miRNAs, chosen for their established relevance to breast cancer, (miR-10b, miR-21, miR-145, miR-155, miR-195, miR-181c, miR-342, miR-16 and let-7a) were detectable in whole blood, serum and plasma samples from breast cancer patients (n=83) as well as healthy controls (n=44) (Table 7). Analysis of miRNA expression levels in whole blood, serum and plasma from a random sample of patients indicated that higher yields of miRNAs by RQ-PCR were obtained from whole blood, compared to either serum or plasma. Based on these findings, whole blood was identified as the preferred medium for investigation of miRNAs in circulation.

TABLE 7
Mean RQ expression levels (SD) of target miRNAs in blood from breast
cancer patients compared with blood from healthy controls.

| | | | | | Mean fold change | |
|---|---|---|---|---|---|---|
| | Breast | | | | in miRNA ex- | |
| | Cancer | | Control | | pression in | |
| | blood | | blood | | breast cancer | |
| Target | samples | | samples | | blood compared | |
| miRNA | (n = 83) | | (n = 44) | | to controls | p-value |
| miR-10b | 1.05 | (3.03) | 0.83 | (0.83) | 1.27 | 0.449 |
| miR-21 | 3.52 | (10.30) | 2.69 | (7.47) | 1.31 | 0.606 |
| miR-145 | 3.58 | (7.29) | 1.65 | (4.14) | 2.17 | 0.062 |
| miR-155 | 2.92 | (6.23) | 1.77 | (4.48) | 1.65 | 0.280 |
| miR-195 | 6.91 | (12.17) | 0.36 | (0.43) | 19.25 | <0.001 |
| let 7a | 5.05 | (24.33) | 0.45 | (0.9) | 11.20 | <0.001 |
| miR-181c | 133.2 | (3.1) | 18.3 | (2.5) | 7.28 | <0.001 |
| miR-342 | 307.9 | (3.7) | 23.4 | (3.2) | 13.16 | <0.001 |

Expression Profiles of Blood miRNAs in Breast Cancer Patients

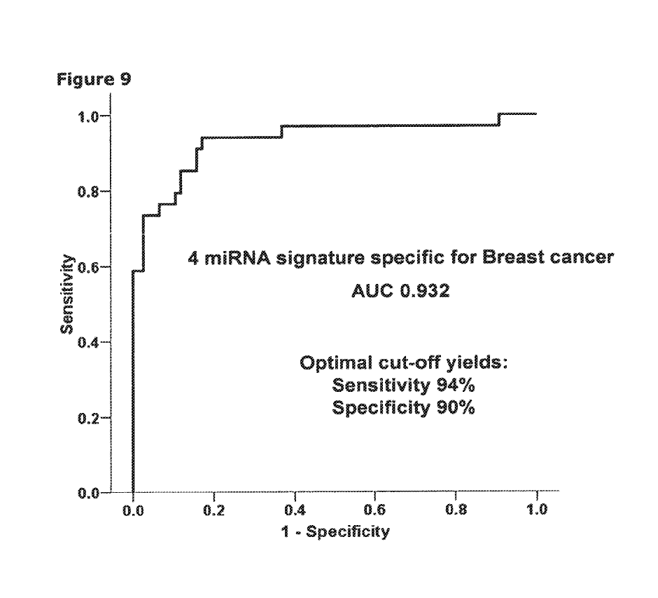

To explore the potential of using circulating miRNAs as novel biomarkers for breast cancer, we investigated the levels of 8 target miRNAs (miR-16 used as endogenous control) in the circulation of 83 consecutive breast cancer patients and compared with those of 44 normal subjects. The levels of four tumor-associated miRNAs (miR-195, miR-181c, miR-342 and let-7a) were significantly higher, on average, in the breast cancer cohort than in healthy controls (p<0.001), corresponding to average fold-changes of 19.2, 7.3, 13.2 and 11.2 respectively (Table 7, FIG. 1). Elevated levels of this 4-miRNA signature increased discriminatory power of this test for breast cancer (all types, including non-invasive breast cancer (DCIS) to 94% (p<0.001), as shown in FIG. 9.

Within this breast cancer cohort, a subset of patients had postoperative blood samples collected (n=29) to assess the effect of curative tumor resection on circulating miRNA levels. Thus it was found that miRNA expression in the blood had decreased significantly to levels comparable with control subjects (p<0.001). Expression of preoperative circulating miR-10b, miR-21, miR-145 and miR-155 did not differ significantly between the breast cancer cohort and controls (p=0.142, 0.587, 0.162, 0.366 respectively).

Relationship of Systemic and Tumor miRNA Profiles

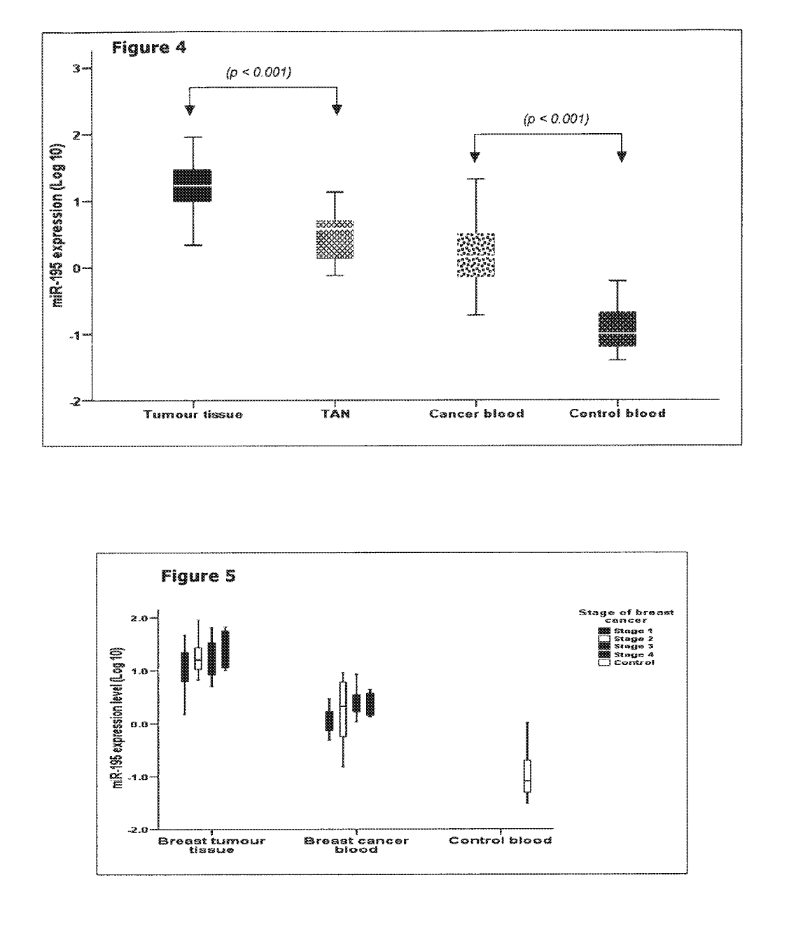

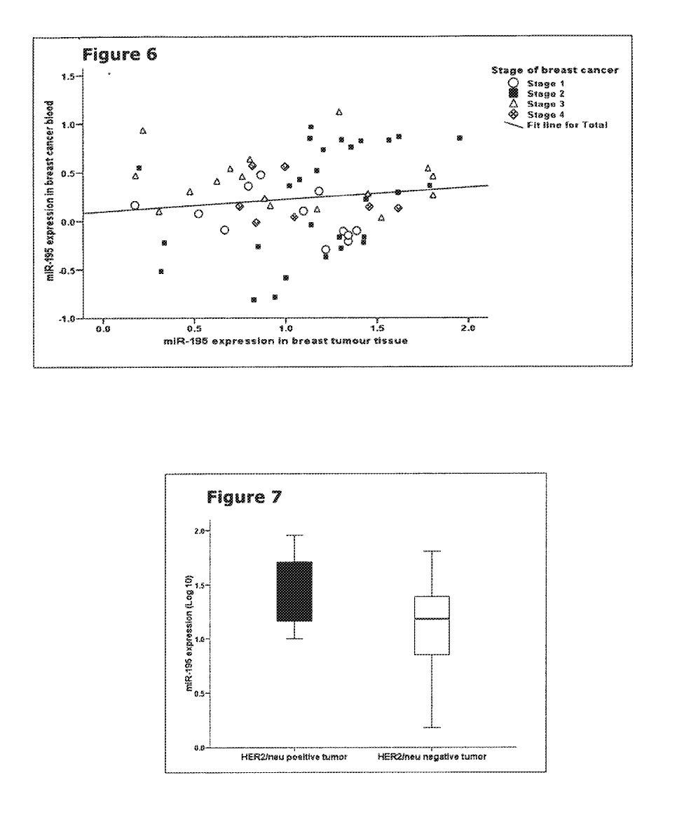

Breast tumor expression of miR-195 was significantly higher compared to that in TAN: 1.23(0.43) vs. 0.49(0.37), p<0.001 (FIG. 4). Tumor miR-195 expression was also significantly higher in Stage IV compared to Stages I & II tumors (p=0.006 and 0.039 respectively ANOVA and Tukey post hoc analysis). There was no significant difference between mean circulating miR-195 expression and the stages of disease, however a similar trend of increasing systemic miR-195 expression with advancing stage of breast cancer was observed (FIG. 5). Controlling for age and stage of disease, miR-195 expression in tumor tissue showed a non-significant positive correlation with circulating miR-195 levels (Pearson's correlation coefficient 0.126, p=0.321) (FIG. 6).

Relationship of Circulating miRNAs to Clinicopathological Parameters

In addition to assessing the relationship of breast tumor and systemic miRNA profiles to the stage of disease, other relevant biopathologic associations of circulating miRNAs were investigated. Lymph node positive patients were found to have significantly lower levels, on average, of circulating let-7a compared to those with node negative disease (n=38 and n=45 respectively, p=0.002).

Higher circulating levels of miR-10b and miR-21 were observed in patients with ER negative disease (n=15), compared to those with ER positive breast cancer (n=68), (p=0.028 and p=0.004 respectively). A potential relationship between circulating miRNA levels, HER2/neu status, and intrinsic subtype was also investigated but no statistically significant difference was identified.

Detection of MiRNA in the Circulation of Patients with Metabolic Disorders

Analysis of the miRNA microarray identified a number of miRNAs which were significantly different between obese and non-obese fat. These miRNAs are all potential therapeutic targets for obesity. Successful validation of the miRNA microarray using RQ-PCR and Taqman miRNA primers and probes was achieved, which confirmed the microarray findings to be accurate. This led to the identification of a signature panel of metabolic miRNAs, which have potentially important roles in obesity and development of the metabolic syndrome.

Quantitation of this signature panel of metabolic miRNAs targets in fat tissue and blood from obese and non-obese patients identified miR-17-5p as an important miRNA, which is significantly decreased in obesity (in both fat and blood). Additionally miR-143 is significantly decreased in obese diabetics (in both fat and blood). MiR-122 expression levels in fat and in blood were shown to correlate positively with total cholesterol and low-density lipoprotein levels. This is the first report of such a finding.

### Discussion:

Circulating miRNAs in breast cancer patients and our results demonstrate that cancer-associated miRNAs in blood can potentially serve as novel non-invasive biomarkers for breast cancer. MiR-195, miR-181c, miR-342 and let-7a are significantly increased in the blood of breast cancer patients in comparison to disease-free control subjects. MiR-195 expression in a similar cohort of breast tumors and TAN specimens shows a similar significant increase in tumor tissue over TAN. When profiling tumor and systemic miR-195 levels according to the stages of invasive breast cancer, similar profiles are evident in both tissue types. In addition, the use of whole blood in preference to plasma or serum for miRNA detection and quantification was successful. Whole blood samples from patient and control subjects were comparable for white cell counts, hemoglobin and hematocrit levels, thereby eliminating potential bias due to cellular and protein components.

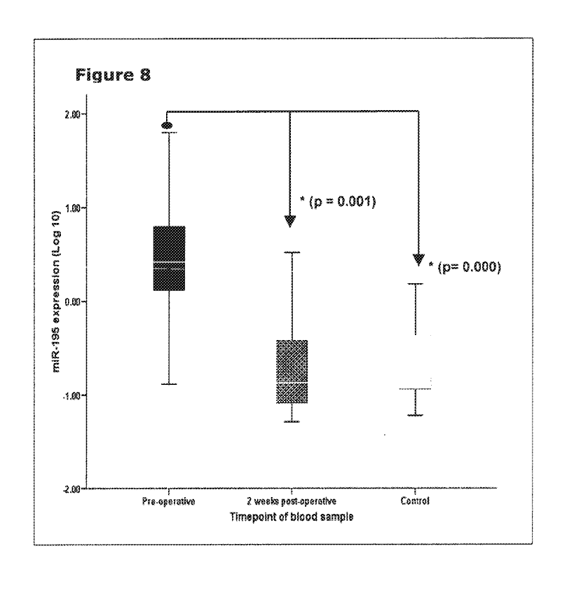

The miRNAs found to be significantly increased in the blood of breast cancer patients, miR-195, miR-181c, miR-342 and let-7a, have previously been described in breast cancer miRNA studies. MiR-195 was reported by Mattie et al to be significantly higher in HER2/neu positive compared to HER2/neu negative breast cancers¹⁶, a finding which was also true for the cohort of 65 invasive breast tumors analysed for miR-195 expression in this study (HER2/neu positive n=20, HER2/neu negative n=45, p=0.002, FIG. 7). Furthermore we observed a significant increase in tumor miR-195 levels in metastatic breast cancers, compared to early stage tumors. This pattern was reflected in the circulation, although to a lesser (non-significant) extent. Interestingly circulating levels of miR-195 decreased significantly by two weeks following tumor resection (FIG. 8) albeit in a small subset of patients. Such observations support the concept of utilising systemic miRNA profiling as a novel and non-invasive biomarker for breast cancer. MiR-181c and miR-342 have both been reported by our group recently to be dysregulated in breast tumours; with levels of miR-342 highest overall in HER2/neu over-expressing and Luminal B tumours, and lowest in the triple negative subgroup. MiR-181c was part of a 5 gene signature associated with HER2/neu positivity.

The finding that let-7a was increased over 5-fold in breast cancer patients was unexpected. Let-7a is well described as having a functional role as a tumor suppressor¹⁸ and has been shown to be down-regulated in many solid organ cancers, including lung, colorectal and gastric cancer^{19-20.} In relation to breast cancer, let-7a in conjunction with miR-16 has been described as a reliable endogenous control for analysis of miRNAs by RQ-PCR in human breast tissue¹³. As endogenous control genes are tissue and organ specific, it is acceptable that a house keeping gene for one tissue type can be investigated as a target gene in another. The finding that let-7a was greatly increased in the blood of breast cancer patients raises an interesting question concerning the origin of circulating miRNAs. Whilst recent blood based miRNA reports, including the present study, clearly show that malignancy alters miRNA levels in the circulation, it is still unknown how tumor associated miRNAs make their way into the bloodstream. Slack et al raised two hypotheses in a recent report²¹, firstly that tumor miRNAs may be present in circulation as a result of tumor cell death and lyses, or alternatively that tumor cells release miRNAs into the tumor micro-environment, where they enter newly formed blood vessels, and thereby make their way into the circulation. Our findings fit generally with the first hypothesis; however it is clear that further studies are needed to gain greater insight into the origin of circulating miRNAs. Further evidence from our investigations support these miRNAs as ideal breast cancer biomarkers. Levels of circulating miRNAs were found to decrease postoperatively, two weeks following curative surgical resection of the breast tumour. Systemic miRNA levels also showed significant correlations with stage of disease; for example miR-195 levels in blood increased as the tumour burden and stage of disease increased, whilst levels of let-7a were highest in patients with non-invasive breast cancer, becoming progressively lower as the stage of invasive disease advanced. Also miR-195 and miR-181c levels were elevated only in breast cancer patients. When miR-195 and miR-181c was measured in the blood of 80 further patients who had just been diagnosed with various cancers other than breast (colon, renal, bladder, prostate, melanoma), levels of miR-195 and miR-181c were elevate only in the breast cancer cohort. Conversely let-7a levels were elevated in almost all cancer patients anlaysed, implicating it as a non-specific general cancer biomarker.

We identified higher circulating levels of miR-21 and miR-10b in patients with ER (oestrogen receptor) negative disease. MiR-21 has been described as an oncomir, and is up-regulated in many solid and hematological cancers. In relation to breast cancer, higher levels of miR-21 have been shown to be associated with advanced disease, poorer prognosis, and lymph node metastasis²²⁻²⁴. However the relationship of tumor miR-21 level to ER status has been inconsistently described; Mattie et al found higher miR-21 levels to be associated with ER positive breast cancer in their study of 20 breast tumor biopsies, 11 of which were ER positive¹⁶. More recently Qian et al showed high miR-21 levels to be associated with estrogen receptor negative disease in a much larger study of 344 breast tumors, 218 of which were known to be ER positive and 120 ER negative²³. Our findings in blood correspond to those of Qian et al in relation to breast tissue. Functional studies have shown that in vitro manipulation of miR-21 expression can alter the responsiveness of ER negative cell lines to hormonal therapies. This further highlights the importance of miR-21 expression in human breast cancer. Although this study did not find circulating miR-21 to differ significantly between breast cancer patients and controls, its association with clinicopathological parameters such as ER status indicates that circulating miR-21 may serve as a prognostic molecular marker for breast cancer and disease progression.

The role of miR-10b in breast cancer has also been addressed with varying conclusions on its precise function. Early studies collectively found miR-10b to be down-regulated in breast tumor compared to normal breast tissue^{14 16}. More recently, Ma et al contested these findings, and reported that miR-10b played a part specifically in the metastatic process but not in primary tumor formation, having found this miRNA to be highly expressed in metastatic breast cancer cells²². To our knowledge, this is the first report of a significant association between miR-10b and the hormonal status of breast cancers. Given that hormone receptor negative status is considered a poor prognostic factor for breast cancer²⁵, our observation that circulating miR-10b is higher in ER negative disease is in keeping with the findings of Ma et al.

### CONCLUSION

The results presented here, showing significantly altered circulating miRNA levels in breast cancer patients compared to healthy individuals, with similar profile for miR-195 in breast tumor tissues compared to TAN, and the associations of particular circulating miRNAs with commonly used prognostic indicators, highlights the potential of these molecules as novel non-invasive biomarkers for breast cancer. Circulating tumor associated miRNAs have the potential to detect breast cancer even in its earliest stages (DCIS), and can differentiate tumors according to tissue type and histological features such as hormone receptor and lymph node status.

Inherent characteristics of miRNAs such as their lower complexity, tissue specific expression profiles, remarkable stability, and ease with which they are amplified and quantified herald these molecules ideal biomarkers to reflect various physiological and pathological states. Methods for effective isolation and quantification of miRNA in blood, or its derivatives, provide the opportunity for these potentially useful clinical biomarkers to be measured non-invasively in the circulation. The invention provides an optimal methodological approach to systemic miRNA detection and quantitation. The results presented here indicate that the optimal extraction technique is a modified version of the TRIZOL® copurification protocol. We have identified whole blood as preferable to serum or plasma for circulating miRNA analysis, and that using higher concentrations (100 ng per reaction) of systemic miRNA in RQ-PCR studies yields superior results compared to lower concentrations. Finally, we have also observed that miR-16 is stably expressed across all analyzed blood samples, including cancers and healthy controls, and thus appears to be a suitable endogenous control for blood based miRNA investigations.

The words “comprises/comprising” and the words “having/including” when used herein with reference to the present invention are used to specify the presence of stated features, integers, steps or components but does not preclude the presence or addition of one or more other features, integers, steps, components or groups thereof.

### EXAMPLE 1

Small RNAs in the circulation were detected as follows:—
1. LYSIS: 3 ml TRI Reagent+200 μl BAN+1 ml whole blood

Ratio of reagent volume to sample volume should always be 3:1

In a 5 ml clear tube place 3.0 ml of TRI Reagent BD supplemented with 200 l of BAN (bromoanisole) and 10 μl of Polyacryl Carrier. Add 1 ml of whole blood.
2. PHASE SEPARATION: homogenate

Split the total volume (>4.2 ml) across 2 round bottomed 2 ml tubes and centrifuge at 14.000 rpm for 15 minutes at 4 C.

It is important to separate phases in the cold (4-10° C.). Centrifugation performed at elevated temperatures may sequester DNA into the aqueous phase. The use of bromoanisole for phase separation improves the quality of isolated RNA and eliminates toxic chloroform and bromochloropropane from the isolation protocol.
3. RNA PRECIPITATION: 1 ml aqueous phase+1 ml isopropanol

Transfer 1 ml of each aqueous phase to a fresh 2 ml round tube. Precipitate RNA from the aqueous phase by mixing with 1 ml isopropanol. Store samples at room temperature for 5 min and centrifuge at 14,000 rpm for 8 minutes at 18° C. RNA precipitate forms a gel-like or white pellet at the bottom of the tube. The 1 ml aliquot of aqueous phases constitutes 80% of the aqueous phase total volume. Leave the remaining 20% undisturbed to prevent accidental collection of DNA from the interphase.
4. RNA WASH: 1 ml 75% ethanol

Remove the supernatant and mix the RNA pellet in 1 ml 75% ethanol by vortexing. Centrifuge the RNA suspension at 14,000 rpm for 5 minutes at 18° C. Repeat this step to improve 260/280 ratio of the RNA.
5. RNA SOLUBILIZATION: 30 μl water per pellet

Remove the ethanol wash and briefly air-dry the RNA pellet for 5 min. It is important not to let the RNA pellet dry completely as this will greatly decrease its solubility. Dissolve the RNA in 30 μl water. Leave the RT for 5 minutes, vortex and spin down 10 sec.
6. RESULTS:

Take a Nanodrop reading at x40 for total RNA and at x33 for miRA. If the duplicate samples are alright, pool the samples, take a fresh NanoDrop reading and reserve 2 μl for Agilent. Store in the appropriately labelled RNA storage tubes (˜60 μl vol).

| Tri Reagant | 750 (μl) | 1 ml | 3 ml |
|---|---|---|---|
| Blood (ml) | 250 | 333.3 | 1 |
| BAN (μl) | 50 | 66.7 | 200 |
| Polyacryl Carrier (μl) | 2-8 | 2.7-10.7 | 8-32 |
| Isopropanol (ml) | 500 | 666.7 | 2 × 1 |
| 75% Ethanol (ml) | 1 | 1.3 | 2 × 1 |
| Water μl | 30 | 30 | 2 × 30 |

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### REFERENCES

1. Calin G A, Dumitru C D, Shimizu M, Bichi R, Zupo S, Noch E, et al. Frequent deletions and down-regulation of micro-RNA genes miR15 and miR16 at 13q14 in chronic lymphocytic leukemia. Proc Natl Acad Sci USA 2002; 99(24):15524-9.
2. Heneghan H M, Miller N, Lowery A J, Sweeney K J, Kerin M J. MicroRNAs as Novel Biomarkers for Breast Cancer. J Oncol 2009; 2009:950201.
3. Piccart-Gebhart M J, Procter M, Leyland-Jones B, Goldhirsch A, Untch M, Smith I, et al. Trastuzumab after adjuvant chemotherapy in HER2-positive breast cancer. N Engl J Med 2005; 353(16):1659-72.
4. Harris L, Fritsche H, Mennel R, Norton L, Ravdin P, Taube S, et al. American Society of Clinical Oncology 2007 update of recommendations for the use of tumor markers in breast cancer. J Clin Oncol 2007; 25(33):5287-312.
5. O'Hanlon D M, Kerin M J, Kent P, Maher D, Grimes H, Given H F. An evaluation of preoperative CA 15-3 measurement in primary breast carcinoma. Br J Cancer 1995; 71(6):1288-91.
6. Uehara M, Kinoshita T, Hojo T, Akashi-Tanaka S, Iwamoto E, Fukutomi T. Long-term prognostic study of carcinoembryonic antigen (CEA) and carbohydrate antigen 15-3 (CA 15-3) in breast cancer. Int J Clin Oncol 2008; 13(5):447-51.
7. Lu J, Getz G, Miska E A, Alvarez-Saavedra E, Lamb J, Peck D, et al. MicroRNA expression profiles classify human cancers. Nature 2005; 435(7043):834-8.
8. Lawrie C H, Gal S, Dunlop H M, Pushkaran B, Liggins A P, Pulford K, et al. Detection of elevated levels of tumour-associated microRNAs in serum of patients with diffuse large B-cell lymphoma. Br J Haematol 2008; 141(5):672-5.
9. Gilad S, Meiri E, Yogev Y, Benjamin S, Lebanony D, Yerushalmi N, et al. Serum microRNAs are promising novel biomarkers. PLoS One 2008; 3(9):e3148.
10. Mitchell P S, Parkin R K, Kroh E M, Fritz B R, Wyman S K, Pogosova-Agadjanyan E L, et al. Circulating microRNAs as stable blood-based markers for cancer detection. Proc Natl Acad Sci USA 2008; 105(30):10513-8.
11. Wang K, Zhang S, Marzolf B, Troisch P, Brightman A, Hu Z, et al. Circulating microRNAs, potential biomarkers for drug-induced liver injury. Proc Natl Acad Sci USA 2009; 106(11):4402-7.
12. Livak K J, Schmittgen T D. Analysis of relative gene expression data using real-time quantitative PCR and the 2(-Delta Delta C(T)) Method. Methods 2001; 25(4):402-08.
13. Davoren P A, McNeill R E, Lowery A J, Kerin M J, Miller N. Identification of suitable endogenous control genes for microRNA gene expression analysis in human breast cancer. BMC Mol Biol 2008; 9:76.
14. Iorio M V, Ferracin M, Liu C G, Veronese A, Spizzo R, Sabbioni S, et al. MicroRNA gene expression deregulation in human breast cancer. Cancer Res 2005; 65(16):7065-70.
15. Lowery A J, Miller N, McNeill R E, Kerin M J. MicroRNAs as prognostic indicators and therapeutic targets: potential effect on breast cancer management. Clin Cancer Res 2008; 14(2):360-5.
16. Mattie M D, Benz C C, Bowers J, Sensinger K, Wong L, Scott G K, et al. Optimized high-throughput microRNA expression profiling provides novel biomarker assessment of clinical prostate and breast cancer biopsies. Mol Cancer 2006; 5:24.
17. Biosystems. RNeasy® Mini Handbook. Qiagen 2006; Fourth Edition(April).
18. Roush S, Slack F J. The let-7 family of microRNAs. Trends Cell Biol 2008; 18(10):505-16.
19. Brueckner B, Stresemann C, Kuner R, Mund C, Musch T, Meister M, et al. The human let-7a-3 locus contains an epigenetically regulated microRNA gene with oncogenic function. Cancer Res 2007; 67(4):1419-23.
20. Yanaihara N, Caplen N, Bowman E, Seike M, Kumamoto K, Yi M, et al. Unique microRNA molecular profiles in lung cancer diagnosis and prognosis. Cancer Cell 2006; 9(3):189-98.
21. Chin L J, Slack F J. A truth serum for cancer—microRNAs have major potential as cancer biomarkers. Cell Res 2008; 18(10):983-4.
22. Ma L, Teruva-Feldstein J, Weinberg R A. Tumor invasion and metastasis initiated by microRNA-10b in breast cancer. Nature 2007; 449(7163):682-88.
23. Qian B, Katsaros D, Lu L, Preti M, Durando A, Arisio R, et al. High miR-21 expression in breast cancer associated with poor disease-free survival in early stage disease and high TGF-beta1. Breast Cancer Res Treat 2008; 117(1):131-40.
24. Wickramasinghe N S, Manavalan T T, Dougherty S M, Riggs K A, Li Y, Klinge C M. Estradiol downregulates miR-21 expression and increases miR-21 target gene expression in MCF-7 breast cancer cells. Nucleic Acids Res 2009.
25. Bauer K R, Brown M, Cress R D, Parise C A, Caggiano V. Descriptive analysis of estrogen receptor (ER)-negative, progesterone receptor (PR)-negative, and HER2-negative invasive breast cancer, the so-called triple-negative phenotype: a population-based study from the California cancer Registry. Cancer 2007; 109(9):1721-28.

1. A method for detecting or screening for breast cancer or a metabolic disease in a patient, comprising;
obtaining a sample comprising whole blood from the patient;
treating the sample with TRIZOL® reagent;
adding 1-bromo-4-methoxybenzyene to the sample with TRIZOL® reagent to provide a mixture;
centrifuging the mixture at at least 12000 g for at least 5 min at less than 10° C., so as to provide an aqueous phase and a non-aqueous phase;
precipitating RNA from the aqueous phase; and
quantifying one or more miRNAs precipitated,
wherein said one or more miRNAs are selected from the group consisting of miR-10b, miR-21, miR145, miR-155, miR-195, miR342, miR181c, let-7a, miR-17-5p, miR-29a, miR-29b, miR-34a, miR-99a, miR-103, miR-122, miR-132, miR-143, and miR-375 biomarkers, and
wherein a statistically significant increase of at least one of said miRNAs relative to a level of said miRNA in control samples from healthy individuals is indicative of breast cancer or metabolic disease in the patient.

2. The method as claimed in claim 1, wherein said one or more miRNAs are selected from the group consisting of miR-10b, miR-21, miR145, miR-155, miR-195, miR342, miR18lc, let-7a, miR-29a, miR-29b, miR-34a, miR-99a, miR-103, miR-122, miR-132, miR-143, and miR-375 biomarkers

3. The method as claimed in claim 1, wherein miR-16 is also quantified as an endogenous control.

4. The method as claimed in claim 1, wherein the method is for detecting or screening for early stage breast cancer in the patient, and said one or more miRNAs are selected from the group consisting of miR-195, miR-342, miR-181c and let-7a, wherein a statistically significant increase of at least one of miR-195, miR-342, miR-181c or let-7a relative to a level of said miRNA in control samples from healthy individuals is indicative of breast cancer in the patient.

5. The method as claimed in claim 1, wherein the method is for detecting or screening for a metabolic disease, and said one or more miRNAs are selected from the group consisting of miR-17-5p, miR-29a, miR-29b, miR-34a, miR-99a, miR-103, miR-122, miR-132, miR-143, miR-145, and miR-375, wherein a statistically significant increase of at least one of miR-17-5p, miR-29a, miR-29b, miR-34a, miR-99a, miR-103, miR-122, miR-132, miR-143, miR-145, or miR-375 relative to a level of said miRNA in control samples from healthy individuals is indicative of metabolic disease in the patient.

6. The method as claimed in claim 5, wherein said one or more miRNAs are selected from the group consisting of miR-29a, miR-29b, miR-34a, miR-99a, miR-103, miR-122, miR-132, miR-143, miR-145, and miR-375.

7. The method as claimed in claim 1, wherein the ratio of whole blood to TRIZOL® reagent is one part whole blood to three parts TRIZOL® reagent.

8. The method as claimed in claim 1 wherein the ratio of 1-bromo-4-methoxybenzyene to the sample comprising the whole blood is in the range of 0.2-0.8 ml 1-bromo-4-methoxybenzyene to 1 ml the sample.

9. The method as claimed in claim 1, wherein the ratio of 1-bromo-4-methoxybenzyene to the sample comprising the whole blood is 0.2 ml 1-bromo-4-methoxybenzyene to 1 ml of the sample.

10. The method as claimed in claim 1, wherein following the addition of 1-bromo-4-methoxybenzyene, a polyacryl carrier is added to the mixture comprising the whole blood, TRIZOL® reagent, and 1-bromo-4-methoxybenzyene.

11. The method as claimed in claim 1, wherein the RNA is precipitated by addition of isopropanol to the aqueous phase.

12. The method as claimed in claim 11, wherein one part isopropanol to one part aqueous phase (v/v) is added.

13. The method as claimed in claim 11, further comprising centrifuging the aqueous phase containing the precipitated RNA for 8 minutes at 12,000 g, at 18 degree Celsius.

14. The method as claimed in claim 1, wherein the precipitated RNA is washed with ethanol.

15. The method as claimed in claim 1, wherein quantification of miRNAs is carried out via NanoDrop® spectrophotometry set at a conversion factor of 33 μg/ml or equivalent spectrophotometry.

16. The method as claimed in claim 1, wherein quantification of miRNAs is carried out via the Agilent quantification method.

17. The method as claimed in claim 1 wherein the method further comprises synthesizing cDNA of said one or more quantified miRNAs.

18. The method as claimed in claim 17, wherein said cDNA is synthesized from 1-1000 ng of the one or more quantified miRNAs.

19. The method as claimed in claim 17, wherein the one or more of the quantified miRNAs is reverse transcribed using stem loop RT primers, specific for each miRNA target and diluted with nuclease-free water to give 50 μM concentration per reaction.

20. The method as claimed in claim 1, wherein miRNA expression levels are relatively quantified by real-time PCR, using an expression level of miR-16 and/or another stably expressed miRNA(s) to normalize an expression level of a target miRNA.
